(19) United States
(12) Patent Application Publication
(10) Pub. No.: US 2008/0182834 A1
(43) Pub. Date: Jul. 31, 2008

(54) Multi-Nuclear Metal Complexes Partially Encapsulated by Cucurbit[7-12]Urils

(75) Inventors: Nial Joseph Wheate, New South Wales (AU); Anthony I. Day, New South Wales (AU); Rodney J. Blanch, Australian Capital Territory (AU); John G. Collins, Australian Capital Territory (AU)

Correspondence Address: STITES & HARBISON PLLC, 1199 NORTH FAIRFAX STREET, SUITE 900, ALEXANDRIA, VA, 22314, US

(73) Assignee: NEWSOUTH INNOVATIONS PTY LIMITED, Sydney (AU)

(21) Appl. No.: 10/586,302

(22) PCT Filed: Jan. 14, 2005
(86) PCT No.: PCT/AU05/00045

§ 371 Date: Nov. 27, 2007

(30) Foreign Application Priority Data
Jan. 15, 2004 (AU) 2004900173

(51) Int. Cl. A61K 31/555; C07D 487/22; A61P 35/00; A61K 31/4188

(52) U.S. Cl. 514/184; 540/460; 514/387

(57) ABSTRACT

The invention provides multi-nuclear metal complexes partially encapsulated by one or more cucurbit[7 to 12]urils or analogues thereof. The invention further relates to methods for treating cancer by administering a multi-nuclear metal complex having anti-tumour activity partially encapsulated by one or more cucurbit[7 to 12]urils or analogues thereof.

### FIELD OF THE INVENTION

The invention relates to multi-nuclear metal complexes partially encapsulated by one or more cucurbit[7 to 12]urils or analogues thereof. The invention further relates to methods for treating cancer by administering a multi-nuclear metal complex having anti-tumour activity partially encapsulated by one or more cucurbit[7 to 12]urils or analogues thereof, and pharmaceutical compositions comprising a multi-nuclear metal complex having anti-tumour activity partially encapsulated by one or more cucurbit[7 to 12]urils or analogues thereof.

### BACKGROUND ART

Cucurbituril is the name given to a cyclic oligomer formed by linking six (6) glycoluril units via methylene bridges. However, the term “cucurbituril” has also been used, and is used in this specification, to refer to a family of compounds (the family including the compound cucurbituril). To avoid confusion, the compound cucurbituril is referred to in this specification as “unsubstituted cucurbit[6]uril”.

Cucurbiturils are a family of cyclic compounds. Cucurbiturils comprise a macrocyclic ring consisting of 4 to 12 units of the formula (C):

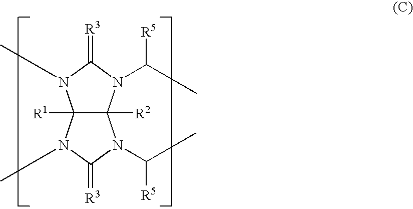

where R¹, R², R³ and R⁵ may be any group and may be different in different units of the formula (C) in the cucurbituril. Cucurbiturils have a central cavity with two openings to the central cavity, the two openings being surrounded by the R³ groups (and R⁵ groups), and the central cavity having a larger diameter than the two openings. Cucurbiturils can encapsulate various compounds, including gases and volatile compounds, within the cavity of the cucurbituril.

Unsubstituted cucurbit[6]uril was first described in the literature in 1905 in a paper by R. Behrend, E. Meyer, F. Rusche, Leibigs Ann. Chem., 399, 1, 1905. The macrocyclic structure of unsubstituted cucurbit[6]uril was first described in 1981 by W. A. Freeman et. al., “Cucurbituril”, J. Am. Chem. Soc., 103 (1981), 7367-7368. Unsubstituted cucurbit[6]uril has a chemical formula of C₃₆H₃₆N₂₄O₁₂ and is a macrocyclic compound having a central cavity.

WO 00/68232 describes the synthesis of various unsubstituted and substituted cucurbit[n]urils. U.S. Pat. No. 6,365,734 also describes the synthesis of various cucurbit[n]urils.

Further cucurbit[n]urils, and methods of preparing cucurbit[n]urils, are described in co-pending international patent application no. PCT/AU2004/001232.

Various cucurbituril analogues have also recently been described. These analogues have the basic structure of a cucurbituril as described above, but wherein one or some of the units of the formula (C) referred to above are replaced with another group, such as an aromatic group (for example, as described in Lagona J. et al, “Cucurbit[n]uril Analogues”, Organic Letters, 2003, Vol 5, No. 20, 3745-3747).

Cisplatin is a mono-nuclear platinum complex having anti-tumour activity. Cisplatin has been used for the treatment of a variety of cancers in humans, including testicular, ovarian, bladder, head and neck, lung and cervical cancers. However, cisplatin has a number of drawbacks. Many human cancers have natural resistance to cisplatin, and of the cancers that initially respond to cisplatin treatment, many later acquire resistance to the drug. The use of cisplatin has been further limited by its toxicity. Other mono-nuclear platinum complexes having anti-tumour activity have been developed, such as carboplatin. Some of these complexes have less toxicity than cisplatin. However, there has been little success in finding mono-nuclear platinum complexes that show activity in cancer cells having a natural resistance to cisplatin.

An entirely new class of platinum(II) complexes having anti-tumour activity has recently been described. These complexes are multi-nuclear platinum(II) complexes containing two, or more, linked platinum centres, where the complex is resistant to chemical breakdown of the complex in a human or animal body such that the complex is delivered as a multi-nuclear complex to the active site (eg a tumour) in the body. Two or more of the platinum centres in the multi-nuclear platinum complex can each bind to DNA, and the complex is thus capable of forming a completely different range of DNA adducts compared to cisplatin and other mono-nuclear platinum complexes. These multi-nuclear platinum complexes are recognized in the art to comprise a unique class of anti-tumour agent. These complexes have distinct chemical and biological properties compared to mono-nuclear platinum complexes such as cisplatin, carboplatin and those described in U.S. Pat. No. 4,225,529. In contrast to mono-nuclear platinum complexes, most multi-nuclear platinum complexes are charged species.

U.S. Pat. No. 4,797,393 describes a bis-platinum(II) complex which is delivered to the active site as a bis-platinum(II) complex. This bis-platinum complex has a bridging diamine or polyamine ligand and has primary or secondary amines or pyridine type nitrogens coordinated to the platinum atoms, as well as two different or identical ligands which may be a halide, sulphate, phosphate, nitrate, carboxylate, substituted carboxylate or dicarboxylate.

U.S. Pat. No. 5,380,897 describes tri-platinum(II) complexes containing three platinum coordination spheres coupled via diamine or triammine bridging agents.

While these bis-platinum(II) and tris-platinum(II) complexes are recognized as effective anti-tumour agents, the use of these complexes to treat cancers has been limited by their toxicity to animals and humans. Other multi-nuclear metal complexes also have anti-tumour or other therapeutic activity, but are also toxic to animals and humans.

It would be desirable to develop a method for reducing the toxicity of bis-platinum(II) and tris-platinum(II) complexes and other multi-nuclear metal complexes.

### DISCLOSURE OF THE INVENTION

The present inventors have found that cucurbit[7 to 12]urils and analogues thereof partially encapsulate multi-nuclear metal complexes. The present inventors have surprisingly found that the multi-nuclear metal complex when encapsulated by a cucurbit[7 to 12]uril or analogue thereof is less toxic to humans and animals than the free complex.

In a first aspect, the present invention provides a multi-nuclear metal complex partially encapsulated by one or more cucurbit[7 to 12]urils or analogues thereof. The metal complex is typically a bi-nuclear or tri-nuclear metal complex.

In some embodiments of the invention, the metal complex is a metal complex of the formula (IIA), (IIB), (IIC) or (IID):

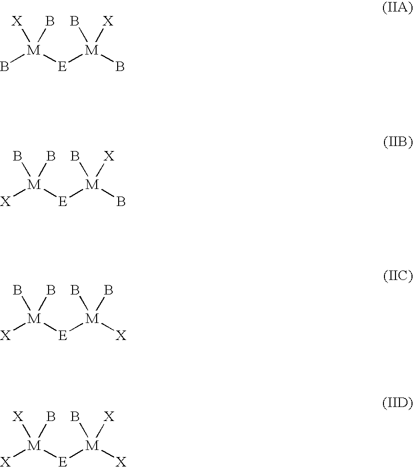

wherein:

each X is independently selected and is a monodentate ligand, or, in the case of formula (IID), the two X groups coordinated to an M atom may each be a monodentate ligand or may together form a dicarboxylate bidentate ligand;

each B is independently selected and is a ligand coordinated to the M atom by a nitrogen atom having a lone pair of electrons;

E is a ligand coordinated to each M atom by a nitrogen atom having a lone pair of electrons; and

each M is independently selected from the group consisting of Pt(II), Pd(II) and Au(II).

In some embodiments, the metal complex is a metal complex of formula (IIIA), (IIIB), (IIIC) or (IIID):

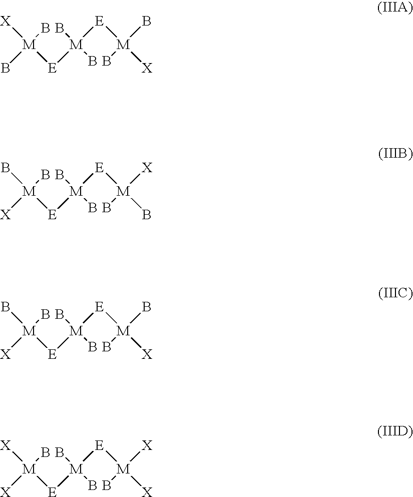

wherein:

each X is independently selected and is a monodentate ligand, or, in the case of formula (IIID), the two X groups coordinated to an M atom may each be a monodentate ligand or may together form a dicarboxylate bidentate ligand;

each B is independently selected and is a ligand coordinated to the M atom by a nitrogen atom having a lone pair of electrons;

each E is independently selected and is a ligand coordinated to each of two M atoms by a nitrogen atom having a lone pair of electrons; and

each M is independently selected from the group consisting of Pt(II), Pd(II) and Au(II).

When X is a monodentate ligand, X is typically selected from the group consisting of halide, sulphate, phosphate (i.e. H₂PO₄⁻ or HPO₄²⁻), nitrate, carboxylate and substituted carboxylate.

Typically B is selected from the group consisting of amine, primary amines, secondary amines, tertiary amines, and groups containing heterocyclic rings containing one or more N atoms.

Typically the metal complex is encapsulated by a cucurbit[7 to 12]uril. Typically the cucurbit[7 to 12]uril is a cucurbituril of the formula (I)

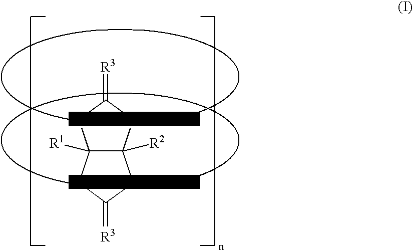

wherein n is an integer from 7 to 12, and wherein for each unit of the formula (B):

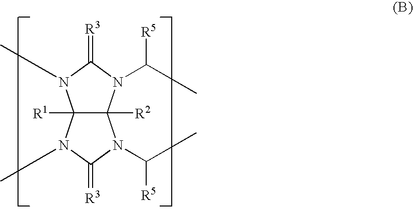

in formula (I),
R¹ and R² may be the same or different and are each a univalent radical, or
R¹, R² and the carbon atoms to which they are bound together form an optionally substituted cyclic group, or
R¹ of one unit of the formula (B) and R² of an adjacent unit of the formula (B) together form a bond or a divalent radical,
each R³ is independently selected from the group consisting of ═O, ═S, ═NR, ═CXZ, ═CRZ, and ═CZ₂, wherein Z is an electron withdrawing group such as —NO₂, —CO₂R, —COR or —CX₃, X is halo and R is H, an optionally substituted straight chain, branched or cyclic, saturated or unsaturated hydrocarbon radical, or an optionally substituted heterocyclyl radical, and
each R⁵ is independently selected from the group consisting of H, alkyl and aryl.

In a second aspect, the present invention provides a method for reducing the in vivo toxicity of a multi-nuclear metal complex, the method comprising forming an association of the metal complex with one or more cucurbit[7 to 12]urils or analogues thereof wherein the metal complex is partially encapsulated by the one or more cucurbit[7 to 12]urils or analogues thereof.

Typically the association of the metal complex with the one or more cucurbit[7 to 12]urils or analogues thereof is formed by contacting the metal complex with the one or more cucurbit[7 to 12]urils or analogues thereof.

In a third aspect, the present invention provides a method for treating cancer in a subject, the method comprising administering to the subject a therapeutically effective amount of a multi-nuclear metal complex having anti-tumour activity partially encapsulated by one or more cucurbit[7 to 12]urils or analogues thereof.

The present invention further provides the use of a multi-nuclear metal complex having anti-tumour activity partially encapsulated by one or more cucurbit[7 to 12]urils or analogues thereof in the manufacture of a medicament for treating cancer in a subject.

The cancer may, for example, be testicular cancer, ovarian cancer, bladder cancer, cancer of the head and neck, lung cancer or cervical cancer. The cancer may be a cancer having resistance to cisplatin.

In a fourth aspect, the present invention provides a pharmaceutical composition comprising a multi-nuclear metal complex having anti-tumour activity partially encapsulated by one or more cucurbit[7 to 12]urils or analogues thereof, and a pharmaceutically acceptable carrier.

### MODES FOR CARRYING OUT THE INVENTION

As used herein, the term “cucurbit[n]uril” refers to a cucurbituril comprising a ring consisting of n units of the formula (C):

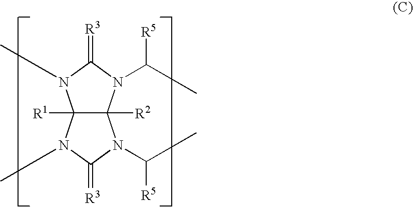

where R¹, R², R³ and R⁵ may be any group, and n is an integer from 4 to 12. Typically, R¹, R², R³ and R⁵ are as defined above for formula (I).

As used herein, the term “unsubstituted cucurbit[n]uril” refers to a cucurbit[n]uril wherein R³ is O and R¹, R² and R⁵ are all H in all of the units of the formula (C) in the cucurbit[n]uril, and the term “substituted cucurbit[n]uril” refers to a cucurbit[n]uril other than an unsubstituted cucurbit[n]uril.

As used herein, an “analogue” of a cucurbit[n]uril refers to a compound having a cyclic structure similar to a cucurbit[n]uril but in which one or some of the units of the formula

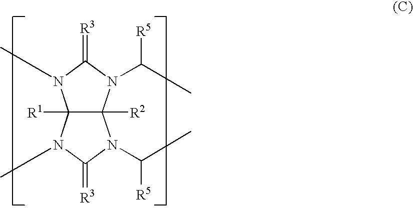

are replaced by another group such as an aromatic group, and wherein the analogue is capable of partially encapsulating multi-nuclear metal complexes.

As used herein, by a multi-nuclear metal complex being partially encapsulated by a cucurbit[7 to 12]uril or analogue thereof, it is meant that part of the metal complex is located within the cavity of the cucurbit[7 to 12]uril or analogue thereof. Typically, the metal complex is reversibly encapsulated by the cucurbit[n]uril or analogue thereof in the sense that under certain conditions the metal complex is released from the cucurbit[n]uril or analogue thereof.

The term “alkyl” used either alone or in a compound word such as “alkylaryl” denotes a straight chain, branched or mono- or poly-cyclic alkyl, preferably C₁₋₃₀ alkyl. Examples of straight chain and branched alkyl include methyl, ethyl, propyl, isopropyl, butyl, isbutyl, sec-butyl, tert-butyl, amyl, isoamyl, sec-amyl, 1,2-dimethylpropyl, 1,1-dimethylpropyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, nonyl, 1-, 2-, 3-, 4-, 5-, 6- or 7-methyloctyl, 1-, 2-, 3-, 4- or 5-ethylheptyl, 1-2- or 3-propylhexyl, decyl, 1-, 2-, 3-, 4-, 5-, 6-, 7- or 8-methylnonyl, 1-, 2-, 3-, 4-, 5- or 6-ethyloctyl, 1-, 2-, 3- or 4-propylheptyl, undecyl 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. Examples of cyclic alkyl include cyclopropyl, cyclobutyl, cyclopentyl, cyclohexyl, cycloheptyl, cyclooctyl, cyclononyl and cyclodecyl and the like.

The term “alkenyl” used either alone or in compound words denotes a straight chain, branched or cyclic alkene, preferably C₂₋₃₀ alkenyl. Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl.

The term “alkoxy” used either alone or in compound words denotes straight chain or branched alkoxy, preferably C₁₋₃₀ alkoxy. Examples of alkoxy include methoxy, ethoxy, n-propyloxy, isopropyloxy and the different butoxy isomers.

The term “aryl” used either alone or in compound words denotes a single, polynuclear, conjugated or fused residue of an aromatic hydrocarbon or aromatic heterocyclic ring system. Examples of aryl include phenyl, naphtyl, pyridyl, furanyl, and the like. When the aryl is a heteroaryl, the aromatic heterocyclic ring system may contain 1 to 4 heteratoms independently selected from N, O and S.

The present invention relates to multi-nuclear metal complexes partially encapsulated by one or more cucurbit[7 to 12]urils or analogues thereof. Such an association of the metal complex and one or more cucurbit[7 to 12]urils or analogues thereof may be referred to as an “association adduct” of the complex with the cucurbit[7 to 12]uril(s) or analogue(s) thereof.

The metal complex is partially encapsulated by the cucurbit[7 to 12]uril or analogue thereof and thus part of the metal complex protrudes from one or both of the openings of the cucurbit[7 to 12]uril or analogue thereof. In some embodiments of the invention, the metal complex is partially encapsulated by two or more cucurbit[7 to 12]urils or analogues thereof.

The metal complex may be any multi-nuclear metal complex. The metal centres in the complex may be the same or different.

Typically, the metal complex is a metal complex of the formula (IIA), (IIB), (IIC), (IID), (IIIA), (IIIB), (IIIC) or (IIID) as defined above. Metal complexes of these formulas have anti-tumour activity. Metal complexes of these formulas are also resistant to chemical breakdown of the multi-nuclear complex in a human or animal body such that when the complex is administered to a human or animal body the complex is delivered to the active site in the body (eg a tumour) as a multi-nuclear metal complex.

However, the present invention is not limited to metal complexes of formula (IIA), (IIB), (IIC), (IID), (IIIA), (IIIB), (IIIC) or (IIID). The metal may for example be another multi-nuclear metal complex such as a complex of the formula:

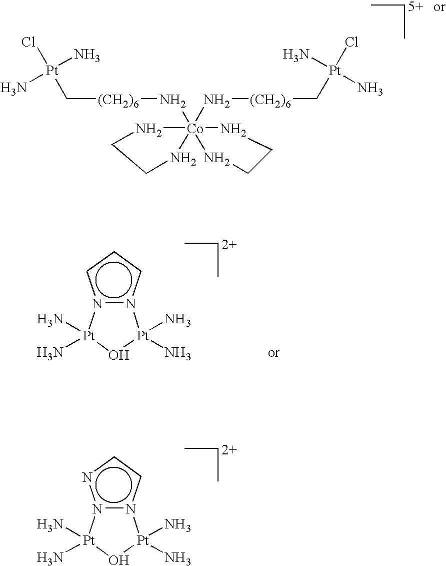

In some embodiments of the invention, the metal complex is a metal complex of the formula (IIA), (IIB), (IIC), (IID), (IIIA), (IIIB), (IIIC) or (IIID), wherein M is Pt(II). Preferably, the metal complex is a metal complex of the formula (IIA) or (IIIA), wherein M is Pt(II). These metal complexes are preferred as it has been found that metal complexes where X and E are in trans-configuration are more effective anti-tumour agents than other isomers of such complexes.

For metal complexes of the formulas (IIA), (IIB), (IIC), (IID), (IIIA), (IIIB), (IIIC) and (IIID), the portion of the complex located within the cavity of the cucurbit[7 to 12]uril or analogue thereof is typically E or part of E.

In formula (IIA), (IIB), (IIC), (IID), (IIIA), (IIIB), (IIIC) or (IIID), when X is a carboxylate or substituted carboxylate, X may be represented by the formula:

CR⁶(C(R⁶)₂)ₘCO₂

wherein m is an integer from 0 to 5 inclusive, the R⁶ groups may be the same or different and may be hydrogen, optionally substituted straight or branched alkyl (eg C₁₋₅ alkyl), optionally substituted aryl, optionally substituted alkylaryl, optionally substituted arylalkyl, optionally substituted alkenyl, optionally substituted alkynyl, optionally substituted cycloalkyl, optionally substituted cycloalkenyl, halogen, pseudohalogen, hydroxy, carbonyl, formyl, nitro, amido, amino, alkoxy, aryloxy and sulfonic acid salts, or the two R⁶ groups in (R⁶)₂ may be combined so that the (R⁶)₂ represents a double bonded oxygen or sulfur. The optional substituents may be selected from aryl, cycloalkyl of 2 to 6 carbon atoms, cycloalkenyl, arylalkyl; halogen, pseudohalogen, hydroxyl, alkoxy, acycloamino or carboxylic acid salts or esters of 1 to 5 carbon atoms.

As used herein, the term “pseudohalogen” has the meaning found at page 560 of “Advanced Inorganic Chemistry” by Cotton and Wilkinson, Interscience Publishers, 1966. That text describes a pseudohalogen as being a molecule consisting of more than two electronegative atoms, which, in the free state, represents halogens. Examples of these molecules are cyanide, cyanate, thiocyanate and azide.

Typically B is selected from the group consisting of ammine (NH₃), primary amines, secondary amines, tertiary amines, and groups containing heterocyclic rings containing one or more N atoms. The heterocyclic ring containing one or more N atoms may be an aromatic group or an aliphatic group. When B is an amine, B may for example be a branched or straight chain alkyl amine (typically C₁₋₅ alkyl amine), aryl amine, arylalkylamine or an alkenyl amine (typically C₁₋₅ alkenyl amine). B may also be a cycloakylamine, polycyclic hydrocarbon amine, nucleoside, nucleotide, pyridine-type nitrogen containing group or an amine with hydroxy, alkoxy (typically C₁₋₅ alkoxy), carboxylic acid or acid ester, nitro or halo substituents.

Preferred primary amines are alkyl-amines of the formula NH₂—R₁₀ where R¹⁰ is a linear or branched C₁₋₅ alkyl, a C₃-C₆ cycloalkyl group (i.e. cyclopropyl, cyclobutyl, cyclopentyl or cyclohexyl), or —CH₂OH.

Preferred secondary amines include alkyl-amines of the formula NH(R¹⁰)₂ wherein each R¹⁰ is independently selected and R¹⁰ is as defined above.

Two B ligands coordinated to a single M atom may be a bidentate ligand such as a diammine. Similarly, E and one or two B ligands may be part of the same tridentate or tetradentate ligand.

E may be any ligand containing two or more N atoms having a lone pair of electrons wherein one such N atom is coordinated with one M atom, and another such N atom is coordinated with another M atom.

E may for example have the formula:

NDG-(C(R⁷)₂)ₙ—(R⁸)ₒ—(C(R⁹)₂)ₚ—NDG

in which n and p are integers from 1 to 6 inclusive and o is 0 or 1;

the R⁷ and R⁹ groups are each independently selected from the group consisting of hydrogen, alkyl (typically C₁₋₅ alkyl), aryl, cycloalkyl, cycloalkenyl, arylalkyl, halogen, pseudohalogen, hydroxy, alkoxy, aryloxy, carboxylic acid ester and carboxylic acid salt, preferably all R⁷ and R⁹ groups are H;

R⁸ is selected from the group consisting of alkyl (eg. C₁₋₅ alkyl), aryl (eg phenyl), amino, alkylamino, diamino of the formula —(NH(CH₂)_{q}NH)— where q is an integer from 1 to 4 inclusive, hydroxyalkyl, alkoxy, sulfur and oxygen; and

each D and G is independently selected from hydrogen, alkyl (typically C₁₋₅ alkyl), aryl, alkylaryl, arylalkyl, alkenyl, cycloalkyl, cycloalkenyl, halogen, pseudohalogen, hydroxy, alkoxy, aryloxy or sulphonic acids or salts thereof. Preferably D and G are hydrogen.

E may for example be spermidine, spermidine doubly methylated at the central N atom, spermine, dipyrazolylmethane or 1,6-hexanediammine.

When B and E are neutral in charge, the overall charge of the metal complex of formula (IIA), (IIB) or (IIC) is typically 2⁺ and the metal complex of formula (IID) is typically neutral. When B and E are neutral in charge, the overall charge of the metal complex of formula (IIIA), (IIIB) or (IIIC) is typically 4⁺ and the metal complex of formula (IIID) is typically 2⁺.

Various multi-nuclear platinum(II) complexes having anti-tumour activity are described in the prior art. For example, various multi-nuclear platinum(II) complexes having anti-tumour activity are described in the article Wheate N J and Collins J G, “Multi-nuclear platinum complexes as anti-cancer drugs”, Coordinated Chemistry Reviews, 241 (2003), 133-145, and in the chapter by Farrell, N in “Platinum-Based Drugs in Cancer Therapy”, Humana Press Totowa, Kellard L. R. and Farrell N. P. (Eds), 2000, pp 321-338, both of which are incorporated herein by reference. The multi-nuclear metal complex used in the present invention may be any of the multi-nuclear platinum(II) complexes described in either of those references.

Examples of specific multi-nuclear platinum(II) complexes having anti-tumour activity described in the prior art include:

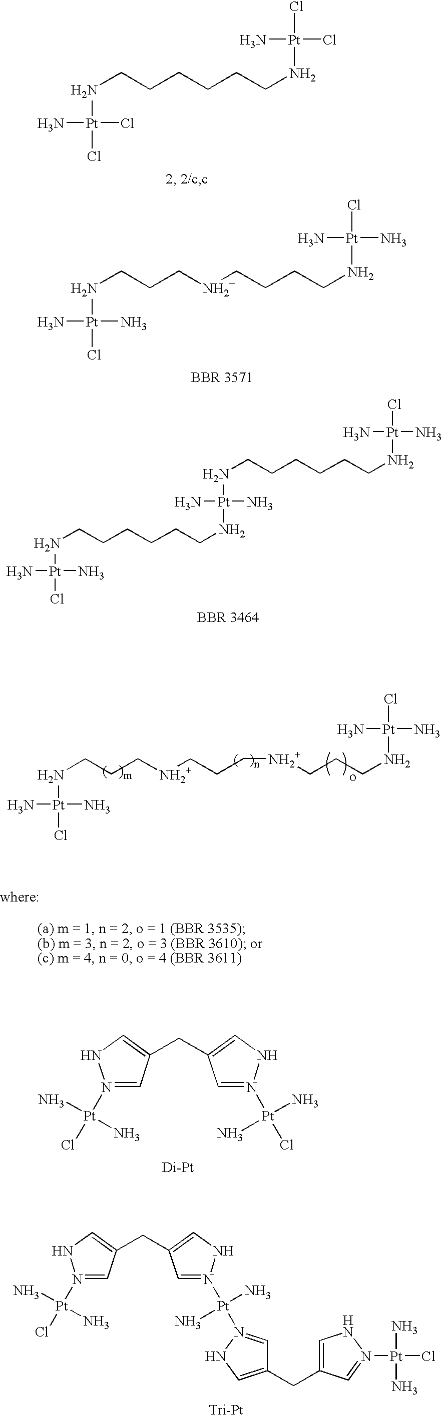

An example of a metal complex of formula (IIA) is:

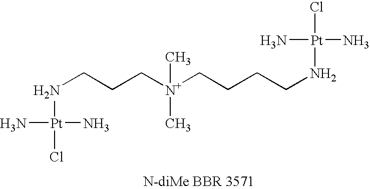

In some embodiments of the present invention, the metal complex is a metal complex of formula (IIA) wherein X is chloride, B is ammine and E is dipyrazolylmethane. This complex, with the counter ion chloride, is known as {trans-diamminechloro(μ-dipyrazolylmethane)platinum(II)}chloride. The complex is referred to below as “Di-Pt”.

In some other embodiments of the invention, the metal complex is a metal complex of formula (IIA) wherein X is chloride, B is ammine and E is spermidine. This complex, with the counter ion chloride, is known as {trans-diamminechloro(μ-spermidine)platinum(II)}chloride. This complex is referred to below as “BBR3571”.

In some embodiments of the invention, the metal complex is a metal complex of formula (IIIA) wherein X is chloride, B is ammine, E is dipyrazolylmethane. This complex with chloride counter ions, is known as {trans-diamminebis{trans-diamminechloro(μ-dipyrazolyl methane)platinum(II)}platinum(II)}chloride. This complex is referred to below as “Tri-Pt”.

In some embodiments of the invention, the metal complex is a metal complex of formula (IIIA) wherein X is chloride, B is ammine and E is 1,6-hexanediammine. This complex, with nitrate counter ions, is known as {trans-diamminebis{trans-diamminechloro(μ-1,6-hexanediamine)platinum(II)}platinum(II)}nitrate. This complex is referred to below as “BBR3464”.

The cucurbit[7 to 12]uril or analogue thereof may be any cucurbit[7 to 12]uril or analogue thereof capable of encapsulating part of the metal complex. Typically the cucurbit[7 to 12]uril is a cucurbit[7 to 12]uril of the formula (I). When the metal complex is partially encapsulated by two or more cucurbit[7 to 12]urils or analogues thereof, the two or more cucurbit[7 to 12]urils or analogues thereof may be the same or different.

Typically, in formula (I), when R¹ and R² are univalent radicals, R¹ and R² are independently selected from the group consisting of —R, —OR, —SR, —NR₂ where each R is independently selected, —NO₂, —CN, —X, —COR, —COX, —COOR,

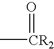

where each R is independently selected,

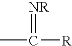

where each R is independently selected, —SeR, —SiR₃ where each R is independently selected, —SR, —SOR,

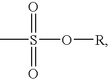

—SO₂R, —S—S—R, —BR₂ where each R is independently selected, —PR₂ where each R is independently selected,

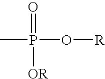

where each R is independently selected,

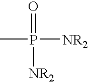

where each R is independently selected, —P⁺R₂ where each R is independently selected and a metal or metal complex, wherein R is H, an optionally substituted straight chain, branched or cyclic, saturated or unsaturated hydrocarbon radical, or an optionally substituted heterocyclyl radical, and X is halo. R may for example be H or a straight chain or branched C₁₋₅ alkyl, or C₂₋₅ alkenyl.

When R¹ and R² are univalent radicals, R¹ and R² may for example be selected from H, an optionally substituted alkyl (e.g. a C₁₋₅ alkyl such as methyl, ethyl, propyl, isopropyl, n-butyl, sec-butyl, isobutyl, tert-butyl, etc), optionally substituted alkenyl, optionally substituted alkynyl, optionally substituted heterocyclyl, optionally substituted aryl (e.g. phenyl, naphthyl, pyridyl, furanyl or thiophenyl), —OR, —SR or —NR₂.

In some embodiments, when R¹ and R² are univalent radicals, R¹ and R² each include less than 30 carbon atoms. R¹ and R² may for example be independently selected from the group consisting of alkyl groups of 1 to 30 carbon atoms, alkenyl groups of 2 to 30 carbon atoms, cyclic hydrocarbon groups of 5 to 30 carbon atoms, cyclic groups of 4 to 30 carbon atoms with one or more heteroatoms such as O, N or S, aryl groups of 6 to 30 carbon atoms, and aryl groups of 5 to 30 carbon atoms with one or more hetero atoms such as O, N or S.

R¹ and R² may for example be an alkoxy group such as methoxy, ethoxy, propyloxy etc. R¹ and R² may also be a hydroxy, halo, cyano, nitro, amino, alkylamino or alkylthio radical.

Examples of optionally substituted cyclic groups formed by R¹, R² and the carbon atoms to which they are bound, include optionally substituted saturated or unsaturated cyclic hydrocarbon groups of 5 to 30 carbon atoms, and optionally substituted saturated or unsaturated cyclic groups of 3 to 30, typically 4 to 30, carbon atoms with one or more heteroatoms such as O, N or S.

The divalent radical which may link R¹ and R² of adjacent units of the formula (B) in the compound of formula (1), may for example, be a divalent optionally substituted straight chain or branched, saturated or unsaturated hydrocarbon radical comprising 1 or more carbon atoms. The divalent radical may consist of or contain one or more heteroatoms such as O, N or S.

When R is an optionally substituted hydrocarbon radical or an optionally substituted heterocyclyl radical, the hydrocarbon radical or the heterocyclyl radical may be substituted by one or more substituents. Similarly, when R¹, R² and the carbon atoms to which they are bound together form an optionally substituted cyclic group, the cyclic group may be substituted by one or more substituents. The optional substituents can be any group and may for example be an optionally substituted alkyl (eg a C₁₋₅ alkyl), an optionally substituted alkenyl (eg. a C₂₋₅ alkyenyl), an optionally substituted alkynyl (eg a C₂₋₅ alkynyl), an optionally substituted heterocyclyl, an optionally substituted aryl, halo (e.g. F, Cl, Br or I), hydroxyl, alkoxyl, carbonyl, acyl halide, nitro, carboxylic acid, carboxylic acid ester, amino, imino, cyano, isocyanate, thiol, thiol-ester, thio-amide, thio-urea, sulfone, sulfide, sulfoxide or sulfonic acid group or a metal or metal complex. The optional substituent may also be a borane, a phosphorous containing group such as a phosphine, alkyl phosphine, phosphate or phosphoramide, a silicon containing group or a selenium containing group.

Typically Z is selected from the group consisting of —NO₂, —CO₂R, —COR and —CX₃, where X is halo (e.g. F, Cl, Br or I) and R is H, alkyl (eg C₁₋₅ alkyl), alkenyl (eg C₂₋₅ alkynyl, alkynyl (eg C₂₋₅ alkynyl), aryl, heteroaryl or saturated or unsaturated heterocyclyl.

The majority of cucurbit[4 to 12]urils prepared to date are cucurbit[4 to 12]urils wherein R³ is O and R⁵ is H in all units of the formula (B) making up the cucurbituril. Accordingly, in some embodiments of the invention, the cucurbit[7 to 12]uril is a cucurbit[7 to 12]uril of formula (I), wherein R³ is O and R⁵ is H in all the units of formula (B) making up the formula (I).

Cucurbit[7 to 12]urils of formula (I) may be prepared as described in WO 00/68232, U.S. Pat. No. 6,365,734 or as described in international patent application no. PCT/AU2004/001232. Analogues of cucurbit[7 to 12]urils may be prepared as described in Lagona J. et al, “Cucurbit[n]uril Analogue”, Organic Letters, 2003, vol 5, no. 20, 3745-3747, incorporated herein by reference.

An association adduct of a multi-nuclear metal complex and a cucurbit[7 to 12]uril or an analogue thereof may be prepared by contacting the metal complex with the cucurbit[7 to 12]uril or analogue thereof. Typically the metal complex is contacted with the cucurbit[7 to 12]uril or analogue thereof by dissolving or suspending the metal complex and the cucurbit[7 to 12]uril or analogue thereof in a solvent, typically water.

The association adduct may for example be formed by the following process:

1 or 2 mole equivalents of cucurbit[7 to 12]uril or analogue thereof (note 1) to the metal complex are either dissolved or suspended in water (note 2), the metal complex is then added, and the mixture stirred at ambient temperature (≦35° C.). The reverse order of addition can be used particularly when the cucurbit[7 to 12]uril or analogues thereof has a low solubility in an aqueous system (note 3). After several hours, all insoluble material is collected or removed by filtration. The formation of the association adduct may be verified by NMR spectroscopy. The aqueous mixture is then freeze-dried (note 4) to give the association adduct as a fine powder.

### Notes

1. The stoichiometry is dependent upon the requirement for a 2:1, a 1:1 or any other required combination of cucurbit[7 to 12]uril or analogue thereof to the multi-nuclear metal complex in the association adduct.
2. In some instances saline solution may be used. Heating to boiling can be used to dissolve cucurbit[7 to 12]uril or analogue thereof in an aqueous system which is then cooled to ambient temperature before the addition of the metal complex.
3. For a cucurbit[7 to 12]uril or analogue thereof which is not very soluble in aqueous systems, organic solvents such as acetonitrile, tetrahydrofuran, trifluoroethanol and formic acid can be added to the aqueous system. Prior to isolation of the association adduct from the aqueous system, the organic solvents are removed in vacuo.
4. When a saline solution is used, the association adduct is not isolated from the solution by freeze drying, but is isolated by crystallization from the saline solution.

The present inventors have found that multi-nuclear metal complexes partially encapsulated by one or more cucurbit[7 to 12]urils or analogues thereof, are less toxic to humans and animals than the unassociated metal complex.

The present invention therefore provides a method for reducing the in vivo toxicity of a multi-nuclear metal complex, the method comprising forming an association of the metal complex with one or more cucurbit[7 to 12]urils or analogues thereof wherein part of the metal complex is encapsulated by the one or more cucurbit[7 to 12]urils or analogues thereof.

Without wishing to be bound by theory, the present inventors believe that the reduction in vivo toxicity of the multi-nuclear metal complex when partially encapsulated by one or more cucurbit[7 to 12]urils or analogues thereof is due to the encapsulation of the metal complex resulting in a decrease in undesirable reactions between the metal complex and compounds in the human or animal body. It is believed that the toxicity of multi-nuclear metal complexes is due, at least in part, to reactions between compounds in the human or animal body, such as thioproteins and/or plasma proteins, and the metal complex, the products of which are believed to induce a toxic reaction. The partial encapsulation of the metal complex by one or more cucurbit[7 to 12]urils or analogues thereof is believed to reduce these reactions, particularly in the blood stream. The reduction in undesirable bio reactions is believed to be due to the cucurbit[7 to 12]uril or analogue thereof hindering reactions between molecules in the body and the multi-nuclear metal complex either sterically by the bulk of the cucurbit[7 to 12]uril or analogue thereof or through a repulsive action by the electronegative portals of the cucurbit[7 to 12]uril or analogue thereof.

Many multi-nuclear metal complexes have anti-tumour activity.

The present invention provides a method for treating cancer in a subject, the method comprising administering to the subject a therapeutically effective amount of a multi-nuclear metal complex having anti-tumour activity partially encapsulated by one or more cucurbit[7 to 12]urils or analogues thereof. The anti-tumour activity of a multi-nuclear metal complex can readily be determined by a person skilled in the art by in vitro screening of the activity of the complex against cancer cell lines. Typically, the multi-nuclear metal complex having anti-tumour activity is a metal complex of formula (IIA), (IIB), (IIC), (IID), (IIIA), (IIIB), (IIIC) or (IIID) as defined above. More typically, the multi-nuclear metal complex is a metal complex of formula (IIA), (IIB), (IIC), (IID), (IIIA), (IIIB), (IIIC) or (IIID) in which M is Pt(II). In some embodiments, the multi-nuclear metal complex having anti-tumour activity is selected from

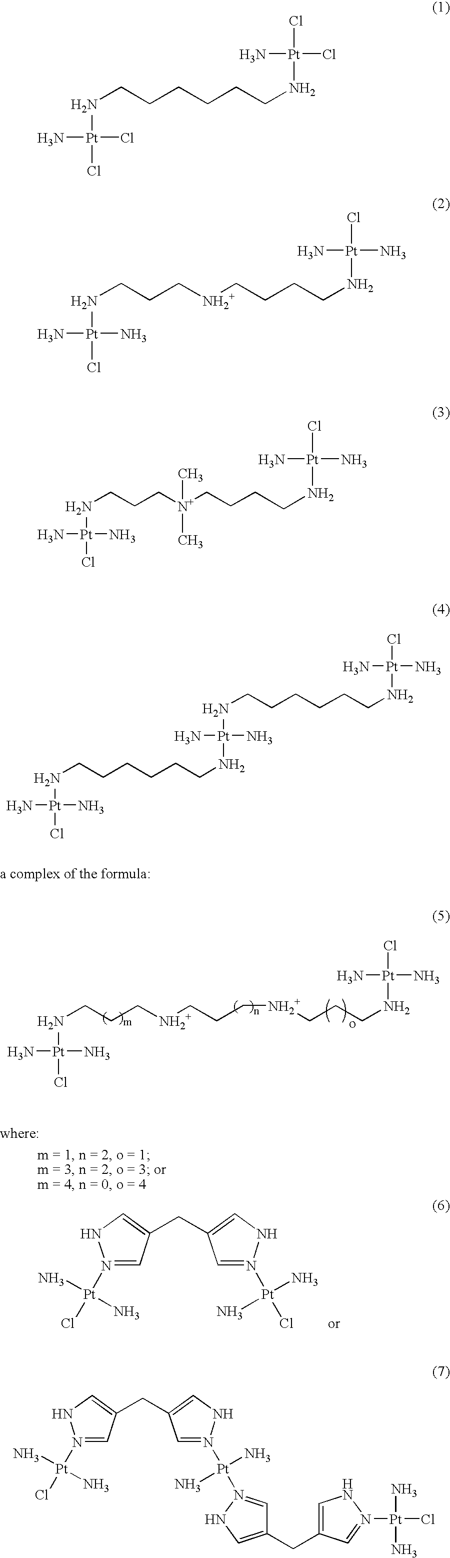

The subject may be a mammal, preferably a human. The subject may be a non-human primate or non-primate such as used in animal model testing. While it is particularly contemplated that the method is suitable for use in medical treatment of humans, it is also applicable to veterinary treatment, including treatment of companion animals such as dogs and cats, and domestic animals such as horses, ponies, donkeys, mules, llama, alpaca, pigs, cattle and sheep, or zoo animals such as primates, felids, canids, bovids, and ungulates.

Suitable mammals include members of the Orders Primates, Rodentia, Lagomorpha, Cetacea, Carnivora, Perissodactyla and Artiodactyla.

As used herein, the term “therapeutically effective amount” refers to an amount effective to yield a desired therapeutic response, for example, to treat cancer by slowing the rate of growth or spread of the cancer cells. The specific “therapeutically effective amount” will, obviously, vary with such factors as the particular condition being treated, the physical condition of the subject, the type of subject being treated, the duration of the treatment, the nature of concurrent therapy (if any), and the specific formulation employed. The association adduct may for example be administered at an effective dose relative to cisplatin taking into account the LD₅₀ value of the association adduct.

The terms “treating”, “treatment” and the like are used herein to mean affecting a subject, tissue or cell to obtain a desired pharmacological and/or physiological effect. The effect may be prophylactic in terms of completely or partially preventing a disease or sign or symptom thereof, and/or may be therapeutic in terms of a partial or complete cure of a disease. “Treating” as used herein covers any treatment of, or prevention of disease, and includes: (a) preventing the disease from occurring in a subject that may be predisposed to the disease, but has not yet been diagnosed as having it; (b) inhibiting the disease, i.e., arresting its development; or (c) relieving or ameliorating the effects of the disease, i.e., cause regression of the effects of the disease.

The association adduct of the metal complex and one or more cucurbit[7 to 12]urils or analogues thereof may additionally be combined with other therapeutic agents to provide an operative combination. It is intended to include any chemically compatible combination of therapeutic agents, as long as the combination does not eliminate the activity of the association adduct. It will be appreciated that the association adduct and the other therapeutic agent may be administered separately, sequentially or simultaneously.

The association adduct can be administered to the subject, orally or parenterally by injection. Administration may be intravenously, intraarterial, intraperitoneally, intramuscularly, subcutaneously, intracavity, transdermally or infusion by, for example, osmotic pump.

The compositions of the present invention comprise at least one association adduct of a multi-nuclear metal complex having anti-tumour activity and one or more cucurbit[7 to 12]urils or analogues thereof, together with one or more pharmaceutically acceptable carriers. The composition may optionally also comprise other therapeutic agents. Compositions of the present invention include those suitable for oral, rectal, nasal, topical (including buccal and sublingual), vaginal or parenteral (including subcutaneous, intramuscular, intravenous and intradermal) administration. The compositions may conveniently be presented in unit dosage form and may be prepared by methods well known in the art of pharmacy. Such methods include the step of bringing into association the active ingredient with the pharmaceutically acceptable carrier and any other components of the composition. In general, the compositions are prepared by uniformly and intimately bringing into association the active ingredient with the carrier and any other components of the composition, and then if necessary shaping the product.

As used herein, a “pharmaceutical carrier” is a pharmaceutically acceptable solvent, suspending agent or vehicle for delivering the active ingredient to the subject. The carrier may be liquid or solid and is selected with the planned manner of administration in mind. The carrier is pharmaceutically “acceptable” in the sense of being not biologically or otherwise undesirable i.e. the carrier may be administered to a subject along with the active ingredient without causing any or a substantial adverse reaction.

A pharmaceutical composition of the present invention for oral use may contain one or more agents selected from the group of sweetening agents, disintegrating agents, flavouring agents, colouring agents, preservatives, lubricants and time delay agents, in order to produce pharmaceutically elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable preservatives include sodium benzoate, vitamin E, alphatocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Pharmaceutical compositions of the present invention in the form of tablets may contain (1) inert diluents, such as calcium carbonate, lactose, calcium phosphate or sodium phosphate; (2) granulating and disintegrating agents, such as corn starch or alginic acid; (3) binding agents, such as starch, gelatin or acacia; and (4) lubricating agents, such as magnesium stearate, stearic acid or talc. The tablets may be uncoated or coated by known techniques to delay disintegration and absorption in the gastrointestinal tract and thereby provide a sustained action over a longer period. For example, a time delay material such as glyceryl monostearate or glyceryl distearate may be employed.

Compositions for parenteral administration include sterile aqueous or non-aqueous solutions, suspensions, and emulsions. Examples of non-aqueous carriers which may be used in such compositions are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers (such as those based on Ringer's dextrose), and the like. Preservatives and other additives may also be present such as, for example, anti-microbials, anti-oxidants, chelating agents, growth factors and inert gases and the like.

Veterinary compositions may be prepared, for example, by methods that are conventional in the art. Examples of such veterinary compositions include those adapted for:

(a) oral administration, e.g. tablets; powders, granules or pellets for admixture with feed stuffs; pastes for application to the tongue;

(b) parenteral administration for example by subcutaneous, intramuscular or intravenous injection, e.g. as a sterile solution or suspension; or (when appropriate) by intramammary injection where a suspension or solution is introduced in the udder via the teat;

(c) topical applications, e.g. as a cream, ointment or spray applied to the skin; or

(d) intravaginally, e.g. as a pessary, cream or foam.

The present invention will now be described below by reference to the following non-limiting examples.

### EXAMPLE 1

Preparation of a Diplatinum Complex Encapsulated by Cucurbit[7 to 12]Uril, 1:1 Association Adduct

Unsubstituted cucurbit[7]uril (1 mole equivalent) was fully dissolved in hot (60-90° C.) 200 mM NaCl solution (150 mL) or H₂O (150 mL). To this was added 1 mole equivalent of {trans-diamminechloro(μ-dipyrazolylmethane)platinum(II)}chloride (Di-Pt) and the solution stirred for 1 hr. Slow evaporation resulted in crystals of the association adduct.

The same procedure was followed using unsubstituted cucurbit[8]uril instead of unsubstituted cucurbit[7]uril, also yielding a white powder.

Similar methods could be used to prepare an association adduct of the metal complex with other unsubstituted or substituted cucurbit[7 to 12]urils, or analogues of cucurbit[7 to 12]urils.

### EXAMPLE 2

Preparation of a Triplatinum Complex Encapsulated by Cucurbit[n]Uril, 1:2 Association Adduct

Approximately 50 mg of either unsubstituted cucurbit[7]uril or unsubstituted cucurbit[8]uril dissolved in water (10 mL) was added to half a molar equivalent of BBR3464 dissolved in water (10 mL). Samples were left to stir for 1 hr at room temperature, after which the solutions were freeze-dried. The samples were analyzed by ¹H NMR spectroscopy showing that the metal complex was encapsulated by the cucurbituril.

Similar methods could also be used to prepare an association adduct of the metal complex with other unsubstituted or substituted cucurbit[7 to 12]urils, or analogues of cucurbit[7 to 12]urils.

### EXAMPLE 3

NMR Analysis of Association Adduct Formation

Aliquots of a solution (5 mM) of either unsubstituted cucurbit[7]uril or unsubstituted cucurbit[8]uril were added directly to an NMR tube containing a dilute solution (1.5 mM) of either BBR3571 or BBR3464 in D₂O, and the ¹H NMR spectrum recorded after each addition showing the metal complex was encapsulated by the cucurbituril.

### EXAMPLE 4

Preparation of Samples for Bioassay

Approximately 50 mg of either unsubstituted cucurbit[7]uril or unsubstituted cucurbit[8]uril dissolved in water (10 mL) was added to equimolar amounts of BBR3571 dissolved in water (10 mL). Samples were left to stir for 1 hr at room temperature, after which the solutions were freeze-dried. The samples were analyzed by ¹H NMR spectroscopy showing that the metal complex was encapsulated by the cucurbituril.

BBR3464/unsubstituted cucurbit[7]uril and BBR3464/unsubstituted cucurbit[8]uril adducts were prepared as in Example 2. The BBR3464/unsubstituted cucurbit[10]uril adduct was prepared as described in Example 5.

### EXAMPLE 5

Sparingly Soluble cucurbit[n]Uril

BBR3464 (6 mg) dissolved in water (2 mL) was added to 5 mg of unsubstituted cucurbit[10]uril, another 4 mL of water added and the suspension stirred overnight. An additional 5 mg of cucurbit[10]uril and 5 mL of water was then added, and the suspension stirred for a further 48 hr. The suspension was then centrifuged and the supernatant freeze-dried. Samples were analyzed by ¹H NMR spectroscopy showing that the metal complex was encapsulated by the cucurbituril.

### EXAMPLE 6

Bioassays in vitro

BBR3571, the association adduct of BBR3571 with unsubstituted cucurbit[7]uril prepared as described above in Example 4, Di-Pt, and the association adduct of Di-Pt with unsubstituted cucurbit[7]uril prepared as described above in Example 1, were tested for cytotoxic activity against L1210 murine leukaemia cells and their matched cisplatin resistant cells L1210/DDP. The tests were carried out in vitro according to the procedures outlined by N. J. Wheate et al in Anti-Cancer Drug Design, 16, 91 (2001), and the results are set out in Table I. The results in Table I are expressed as the IC₅₀ which represents the minimum concentration of the complex or association adduct required to inhibit cell growth by 50%.

TABLE I

| | | IC₅₀ | | |
|---|---|---|---|---|
| COMPLEX | CUCURBITURIL | | L1210 | L1210/DDP |

| | | | | | |
|---|---|---|---|---|---|
| BBR3571 | NIL | 11.5 | nM | 7.5 | nM |
| BBR3571 | 7 | 11.5 | nM | 9 | nM |
| Di-Pt | NIL | 3.8 | μM | 8.8 | μM |
| Di-Pt | 7 | 2.6 | μM | 16.5 | μM |

As the association adducts gave similar values to the free complex, the association adducts are considered effective anti-cancer agents against these leukaemia cell lines. The general cytotoxic activity of BBR3571 and Di-Pt was thus maintained in the association adducts of BBR3571 and Di-Pt with unsubstituted cucurbit[7]uril.

The ability to adjust the cytotoxic activity of the platinum complexes by forming an association adduct with a cucurbit[7 to 10]uril is demonstrated in Table II, following the procedures outlined above using the association adduct of BBR3464 with unsubstituted cucurbit[7]uril, unsubstituted cucurbit[8]uril or unsubstituted cucurbit[10]uril prepared as described in Examples 4 and 5. The in vitro tests demonstrate that the cytotoxicity of complex BBR3464 was reduced by decreasing the size of the encapsulating cucurbit[n]uril.

TABLE II

IC₅₀ (μM)

| COMPLEX | CUCURBITURIL | L1210 | L1210/DDP |
|---|---|---|---|
| BBR3464 | NIL | 57 nM | 24.5 nM |
| BBR3464 | 10 | 0.7 | 0.2 |
| BBR3464 | 8 | 6.6 | 1.4 |
| BBR3464 | 7 | >37.5 | >37.5 |

### EXAMPLE 7

Bioassays in vivo

### A. Maximum Tolerated Dose (MTD)

Platinum complex BBR3571 and the association adduct of BBR3571 and unsubstituted cucurbit[7]uril were tested in vivo in female balb/c nude mice.

The results of these tests showed that the association adduct had a maximum tolerated dose (MTD) of BBR3571 1.7 times higher than the free complex, when delivered intravenously in saline solution.

TABLE III

| | | MTD | Drug |
|---|---|---|---|
| COMPLEX | CUCURBIT[N]URIL | (mg/kg) | Equivalence |
| BBR3571 | NIL | 0.1 | 1 |
| BBR3571 | 7 | 0.45 | 1.7 |

The maximum tolerated dose of free platinum complex BBR3571 is 0.1 mg/kg compared to 0.45 mg/kg for the cucurbit[7]uril/BBR3571 association adduct.

B. Cytotoxic Activity of the Free Metal Complex Versus the Association Adduct in vivo

The cytotoxic activity of the association adduct of BBR3571 and unsubstituted cucurbit[7]uril at a drug equivalence of 1 (equimolar amount) was compared to the free metal complex. The experiment was limited to the MTD of the free metal complex. Female balb/c nude mice were inoculated subcutaneously on the flank with cells from the 2008 ovarian carcinoma cell line. Once the tumours had reached a volume of approximately 100 mm³ the mice were randomized into groups and administered either a saline solution of BBR3571 at MTD or a saline solution of the association adduct of unsubstituted cucurbit[7]uril and BBR3571 in an equimolar amount (0.27 mg/kg of the association adduct). The controls were administered either as saline or a saline solution of unsubstituted cucurbit[7]uril. Doses were administered on days 0, 4 and 8. The results are shown in Table IV.

TABLE IV

| | CURCURBIT[N]- | DOSE | DRUG | TGI | |
|---|---|---|---|---|---|
| COMPLEX | URIL | (mg/kg) | Equivalence | (%)^{a} | GDI^{b} |
| BBR3571 | NIL | 0.10 | 1 | 48.5 | 1.6 |
| BBR3571 | 7 | 0.27 | 1 | 44.9 | 1.7 |

^{a}Tumour Growth Index (TGI) defined as 100 minus (the median relative tumour volume of the treated group of mice divided by the median relative tumour volume of the control group of mice multiplied by 100).
^{b}Growth Delay Index (GDI) defined as the median growth delay of the treated tumours divided by the median growth delay of the control (untreated) tumours.

The free complex and association adduct show comparable activity at a drug equivalence of 1 for both.

### EXAMPLE 8

General ¹H NMR Spectra of Cucurbit[n]Uril/Metal Complex Association Adducts

The association adducts of BBR3571 with unsubstituted cucurbit[7]uril or unsubstituted cucurbit[8]uril prepared in Example 4, Di-Pt with unsubstituted cucurbit[7]uril prepared in Example 1, Tri-Pt with unsubstituted cucurbit[7]uril prepared by a similar method to that described in Example 2, BBR3464 with unsubstituted cucurbit[7]uril or unsubstituted cucurbit[8]uril prepared in Example 2, and BBR3464 with unsubstituted cucurbit[10]uril prepared in Example 5 were analyzed by ¹H NMR spectroscopy and results are shown in Table V.

The characteristic shielding effect of the cavity of cucurbit[n]uril shows that in most examples the proton resonances of the metal complex as an association adduct are shifted up field (indicated by a minus sign) when compared to samples of the free metal complex. This shows that the linking group E is bound within the cavity of cucurbit[n]uril, and thus confirming that the metal complex is partially encapsulated by the cucurbituril.

### EXAMPLE

1. In BBR3571

E=NH₂CH₂CH₂CH₂NH₂⁺CH₂CH₂CH₂CH₂NH₂

a b c d*c*b*a*
2. In complexes Di-Pt and Tri-Pt E=

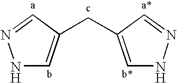

3. In BBR3464 E=NH₂CH₂CH₂CH₂CH₂CH₂CH₂NH₂

a b c c*b*a*

TABLE V
Table of ¹H NMR resonance shifts. A comparison of the difference in
chemical shift between the protons of the free metal complex and the
same protons as a cucurbit[n]uril/metal complex adduct.

| | Chemical shift difference | | | |
|---|---|---|---|---|
| Structure/ | | Cucurbit[7] | Cucurbit[8] | Cucurbit[10] |
| Example | Protons | uril | uril | uril |
| BBR3571 | a | +0.20 | 0.02 | — |
| | a* | −0.73 | −0.50 | — |
| | b | +0.11 | −0.28 | — |
| | b* | −1.03 | −0.80 | — |
| | c | −0.02 | −0.31 | — |
| | c* | −1.03 | −0.72 | — |
| | d* | −0.96 | −0.72 | — |
| Di-Pt | a = a* | −0.37 | — | — |
| | b = b* | −1.49 | — | — |
| | c | −0.88 | — | — |
| BBR3464 | a | −0.38 | −0.50 | −0.34 |
| | b | −0.25 | −0.30 | −0.45 |
| | c | −1.00 | −0.72 | −0.59 |
| | c* | −1.00 | −0.61 | −0.44 |
| | b* | −0.75 | −0.61 | −0.21 |
| | a* | −0.75 | −0.61 | −0.25 |
| Tri-Pt | a = a* | −0.53 | — | — |
| | b = b* | −1.54 | — | — |
| | c | −0.84 | — | — |

The present inventors have found that cucurbit[7 to 12]urils and analogues thereof partially encapsulate multi-nuclear metal complexes, and that the resultant association adducts are less toxic to the human or animal body than the free metal complex. In view of the size of multi-nuclear metal complexes, they are not fully encapsulated within the cucurbit[7 to 12]uril or analogue thereof. Nevertheless, it has been surprisingly found by the present inventors that when a multi-nuclear metal complex is partially encapsulated by one or more cucurbit[7 to 12]urils or analogues thereof, the resultant association adduct is less toxic to humans and animals than the free complex, and thus higher doses of the complex can be administered to a human or animal as part of an association adduct with one or more cucurbit[7 to 12]urils or analogues thereof than can be administered as the free complex. Association adducts of multi-nuclear metal complexes having anti-tumour activity and one or more cucurbit[7 to 12]urils or analogues thereof may be used for the treatment of conditions which can be treated using the metal complex.

The partial encapsulation of the multi-nuclear metal complex by one or more cucurbit[7 to 12]urils or analogues thereof may also provide a number of other advantages. For example, the cucurbit[7 to 12]uril or analogue thereof may provide for better delivery or targeting of the multi-nuclear metal complex to the desired site in the body. Targeting could be achieved through appropriate substituents on the cucurbit[7 to 12]uril or analogue thereof. For example, a lipophilic group on the cucurbit[7 to 12]uril or analogue thereof may assist in the delivery of the multi-nuclear metal complex to lipophilic tumours and cancers such as those of the liver. Further, cucurbit[7 to 12]urils or analogues thereof attached to or incorporated into polymers could provide a means for delivery of the multi-nuclear metal complex over extended periods of time. In addition, different cucurbit[7 to 12]urils may have different binding capacities to the multi-nuclear metal complex, and thus could be used to provide a particular rate of release of the multi-nuclear metal complex over time.

As will be apparent to a person skilled in the art, the multi-nuclear metal complexes partially encapsulated by one or more cucurbit[7 to 12]urils or analogues thereof of the present invention have a wide range of applications in the medical and veterinary fields. The method for reducing the in vivo toxicity of a multi-nuclear metal complex of the present invention can, for example, be used to reduce the toxicity of pharmaceutically active multi-nuclear metal complexes, including multi-nuclear metal complexes having anti-tumour activity.

It will be appreciated by the person skilled in the art that numerous variations and/or modifications may be made to the invention as described in the examples without departing from the spirit or scope of the invention as broadly described. The embodiments described in the examples are therefore to be considered in all respects as illustrative and not restrictive.

1.-26. (canceled)

27. A multi-nuclear metal complex partially encapsulated by one or more cucurbit[7 to 12]urils or analogues thereof.

28. The multi-nuclear metal complex partially encapsulated by one or more cucurbit[7 to 12]urils or analogues thereof as claimed in claim 27, wherein the metal complex is a bi-nuclear or tri-nuclear metal complex.

29. The multi-nuclear metal complex partially encapsulated by one or more cucurbit[7 to 12]urils or analogues thereof as claimed in claim 27, wherein the metal complex is a metal complex of the formula (IIA), (IIB), (IIC) or (IID): 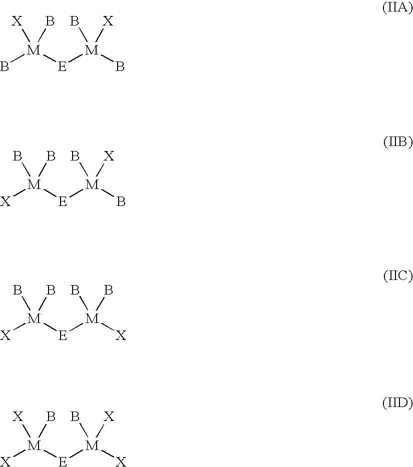
wherein:
each X is independently selected and is a monodentate ligand, or, in the case of formula (IID), the two X groups coordinated to a M atom may each be a monodentate ligand or may together form a dicarboxylate bidentate ligand;
each B is independently selected and is a ligand coordinated to the M atom by a nitrogen atom having a lone pair of electrons;
E is a ligand coordinated to each M atom by a nitrogen atom having a lone pair of electrons; and
each M is independently selected from the group consisting of Pt(II), Pd(II) and Au(II).

30. The multi-nuclear metal complex partially encapsulated by one or more cucurbit[7 to 12]urils or analogues thereof as claimed in claim 29, wherein X is a monodentate ligand selected from the group consisting of halide, sulphate, phosphate, nitrate, carboxylate and substituted carboxylate.

31. The multi-nuclear metal complex partially encapsulated by one or more cucurbit[7 to 12]urils or analogues thereof as claimed in claim 29, wherein B is selected from the group consisting of ammine, primary amines, secondary amines, tertiary amines, and groups containing heterocyclic rings containing one or more N atoms.

32. The multi-nuclear metal complex partially encapsulated by one or more cucurbit[7 to 12]urils or analogues thereof as claimed in claim 29, wherein M is Pt(II).

33. The multi-nuclear metal complex partially encapsulated by one or more cucurbit[7 to 12]urils or analogues thereof as claimed in claim 27, wherein the metal complex is a metal complex of formula (IIIA), (IIIB), (IIIC) or (IIID): 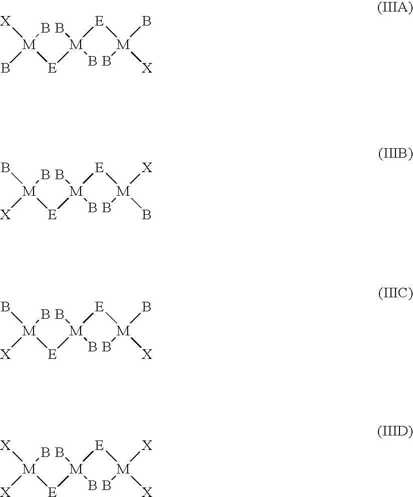
wherein:
each X is independently selected and is a monodentate ligand, or, in the case of formula (IIID), the two X groups coordinated to a M atom may each be a monodentate ligand or may together form a dicarboxylate bidentate ligand;
each B is independently selected and is a ligand coordinated to the M atom by a nitrogen atom having a lone pair of electrons;
each E is independently selected and is a ligand coordinated to each of two M atoms by a nitrogen atom having a lone pair of electrons; and
each M is independently selected from the group consisting of Pt(II), Pd(II) and Au(II).

34. The multi-nuclear metal complex partially encapsulated by one or more cucurbit[7 to 12]urils or analogues thereof as claimed in claim 33, wherein X is a monodentate ligand selected from the group consisting of halide, sulphate, phosphate, nitrate, carboxylate and substituted carboxylate.

35. The multi-nuclear metal complex partially encapsulated by one or more cucurbit[7 to 12]urils or analogues thereof as claimed in claim 33, wherein B is selected from the group consisting of ammine, primary amines, secondary amines, tertiary amines, and groups containing heterocyclic rings containing one or more N atoms.

36. The multi-nuclear metal complex partially encapsulated by one or more cucurbit[7 to 12]urils or analogues thereof as claimed in claim 33, wherein M is Pt(II).

37. The multi-nuclear metal complex partially encapsulated by one or more cucurbit[7 to 12]urils or analogues thereof as claimed in claim 33, wherein the metal complex is selected from:

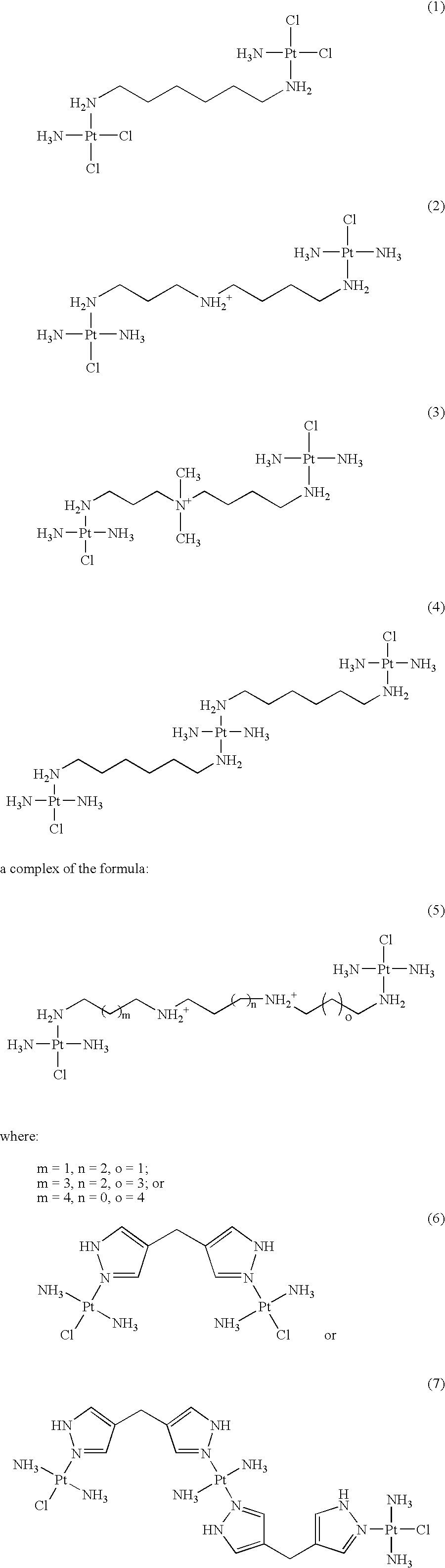

38. The multi-nuclear metal complex partially encapsulated by one or more cucurbit[7 to 12]urils or analogues thereof as claimed in claim 27, wherein the cucurbit[7 to 12]uril is a cucurbituril of the formula (I) 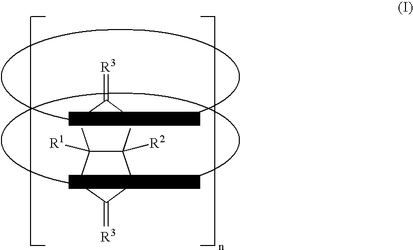
wherein n is an integer from 7 to 12, and wherein
for each unit of the formula (B): 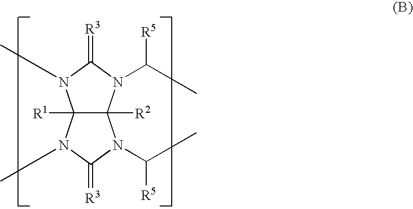
in formula (I),
R¹ and R² may be the same or different and are each a univalent radical, or
R¹, R² and the carbon atoms to which they are bound together form an optionally substituted cyclic group, or
R¹ of one unit of the formula (B) and R² of an adjacent unit of the formula (B) together form a bond or a divalent radical,
each R³ is independently selected from the group consisting of ═O, ═S, ═NR, ═CXZ, ═CRZ, and ═CZ₂, wherein Z is an electron withdrawing group, X is halo and R is H, an optionally substituted straight chain, branched or cyclic, saturated or unsaturated hydrocarbon radical, or an optionally substituted heterocyclyl radical, and each R⁵ is independently selected from the group consisting of H, alkyl and aryl.

39. A method for reducing the in vivo toxicity of a multi-nuclear metal complex, the method comprising forming an association of the metal complex with one or more cucurbit[7 to 12]urils or analogues thereof wherein the metal complex is partially encapsulated by the one or more cucurbit(7 to 12]urils or analogues thereof.

40. The method as claimed in claim 39, wherein the metal complex is a metal complex of the formula (IIA), (IIB), (IIC) or (IID): 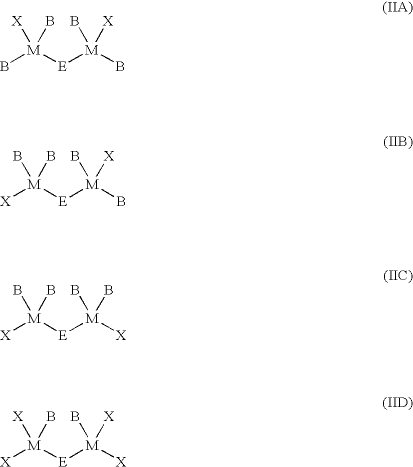
wherein:
each X is independently selected and is a monodentate ligand, or, in the case of formula (IID), the two X groups coordinated to a M atom may each be a monodentate ligand or may together form a dicarboxylate bidentate ligand;
each B is independently selected and is a ligand coordinated to the M atom by a nitrogen atom having a lone pair of electrons;
E is a ligand coordinated to each M atom by a nitrogen atom having a lone pair of electrons; and
each M is independently selected from the group consisting of Pt(II), Pd(II) and Au(II).

41. The method as claimed in claim 40, wherein X is a monodentate ligand selected from the group consisting of halide, sulphate, phosphate, nitrate, carboxylate and substituted carboxylate.

42. The method as claimed in claim 40, wherein B is selected from the group consisting of ammine, primary amines, secondary amines, tertiary amines, and groups containing heterocyclic rings containing one or more N atoms.

43. The method as claimed in claim 40, wherein M is Pt(II).

44. The method as claimed in claim 39, wherein the metal complex is a metal complex of formula (IIIA), (IIIB), (IIIC) or (IIID): 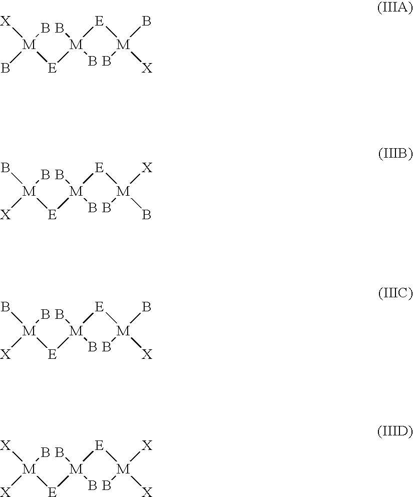
wherein:
each X is independently selected and is a monodentate ligand, or, in the case of formula (IIID), the two X groups coordinated to a M atom may each be a monodentate ligand or may together form a dicarboxylate bidentate ligand;
each B is independently selected and is a ligand coordinated to the M atom by a nitrogen atom having a lone pair of electrons;
each E is independently selected and is a ligand coordinated to each of two M atoms by a nitrogen atom having a lone pair of electrons; and
each M is independently selected from the group consisting of Pt(II), Pd(II) and Au(II).

45. The method as claimed in claim 44, wherein X is a monodentate ligand selected from the group consisting of halide, sulphate, phosphate, nitrate, carboxylate and substituted carboxylate.

46. The method as claimed in claim 44, wherein B is selected from the group consisting of ammine, primary amines, secondary amines, tertiary amines, and groups containing heterocyclic rings containing one or more N atoms.

47. The method as claimed in claim 44, wherein M is Pt(II).

48. The method as claimed in claim 39, wherein the metal complex is selected from:

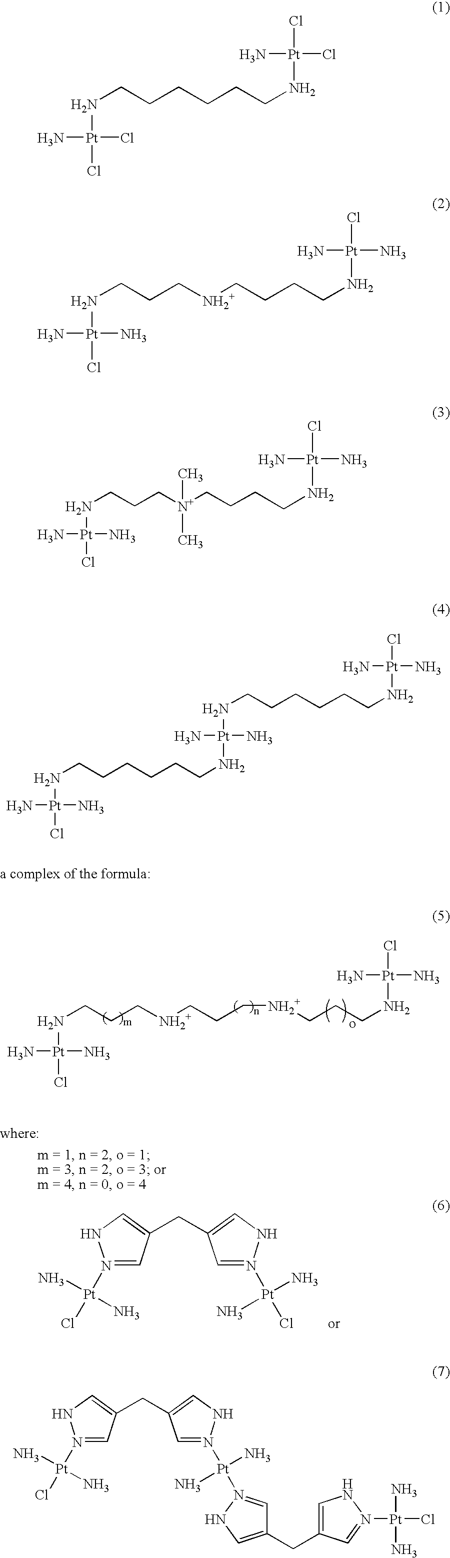

49. A method for treating cancer in a subject, the method comprising administering to the subject a therapeutically effective amount of a multi-nuclear metal complex having anti-tumour activity partially encapsulated by one or more cucurbit[7 to 12]urils or analogues thereof.

50. The method as claimed in claim 49, wherein the metal complex is a metal complex of the formula (IIA), (IIB), (IIC) or (IID): 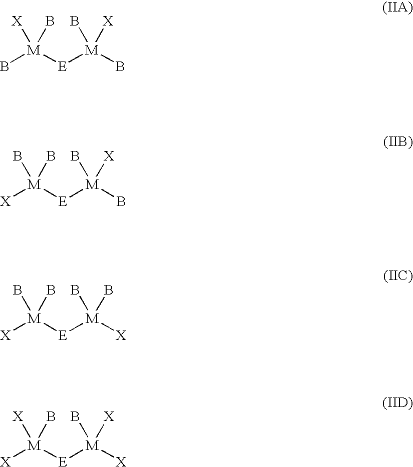
wherein:
each X is independently selected and is a monodentate ligand, or, in the case of formula (IID), the two X groups coordinated to a M atom may each be a monodentate ligand or may together form a dicarboxylate bidentate ligand;
each B is independently selected and is a ligand coordinated to the M atom by a nitrogen atom having a lone pair of electrons;
E is a ligand coordinated to each M atom by a nitrogen atom having a lone pair of electrons; and
each M is independently selected from the group consisting of Pt(II), Pd(II) and Au(II).

51. The method as claimed in claim 50, wherein X is a monodentate ligand selected from the group consisting of halide, sulphate, phosphate, nitrate, carboxylate and substituted carboxylate.

52. The method as claimed in claim 50, wherein B is selected from the group consisting of ammine, primary amines, secondary amines, tertiary amines, and groups containing heterocyclic rings containing one or more N atoms.

53. The method as claimed in claim 50, wherein M is Pt(II).

54. The method as claimed in claim 49, wherein the metal complex is a metal complex of formula (IIIA), (IIIB), (IIIC) or (IIID): 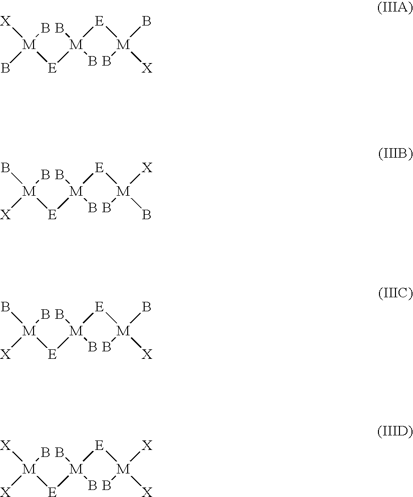
wherein:
each X is independently selected and is a monodentate ligand, or, in the case of formula (IIID), the two X groups coordinated to a M atom may each be a monodentate ligand or may together form a dicarboxylate bidentate ligand;
each B is independently selected and is a ligand coordinated to the M atom by a nitrogen atom having a lone pair of electrons;
each E is independently selected and is a ligand coordinated to each of two M atoms by a nitrogen atom having a lone pair of electrons; and
each M is independently selected from the group consisting of Pt(II), Pd(II) and Au(II).

55. The method as claimed in claim 54, wherein X is a monodentate ligand selected from the group consisting of halide, sulphate, phosphate, nitrate, carboxylate and substituted carboxylate.

56. The method as claimed in claim 54, wherein B is selected from the group consisting of ammine, primary amines, secondary amines, tertiary amines, and groups containing heterocyclic rings containing one or more N atoms.

57. The method as claimed in claim 54, wherein M is Pt(II).

58. The method as claimed in claim 49, wherein the metal complex is selected from:

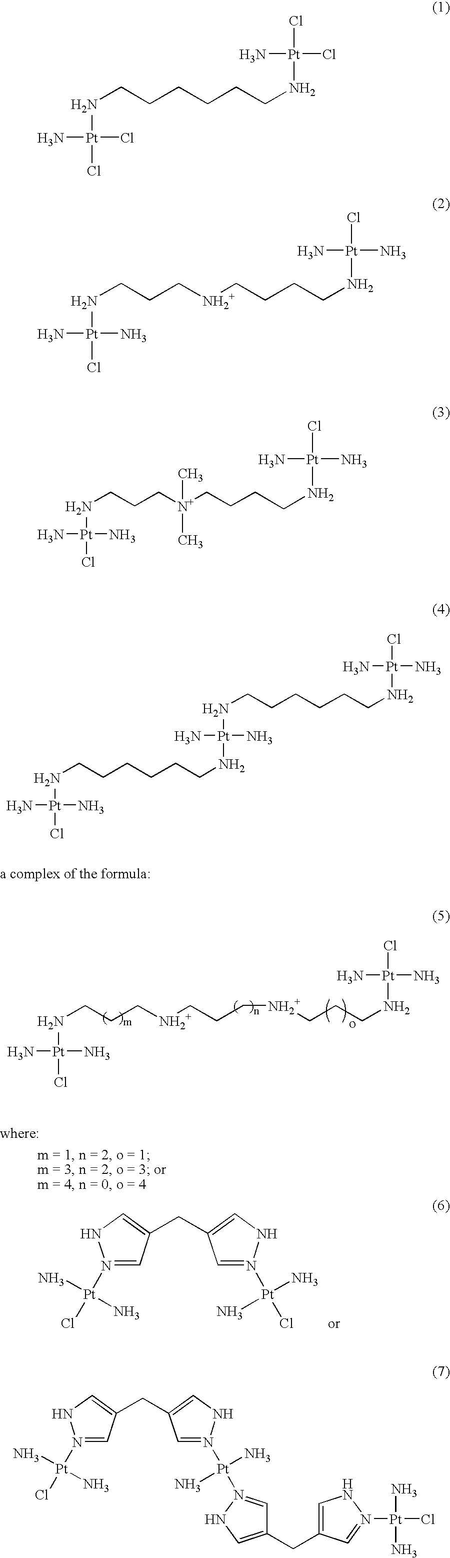

59. A pharmaceutical composition comprising a multi-nuclear metal complex having anti-tumour activity partially encapsulated by one or more cucurbit[7 to 12]urils or analogues thereof, and a pharmaceutically acceptable carrier.

60. The composition as claimed in claim 59, wherein the metal complex is a metal complex of the formula (IIA), (IIB), (IIC) or (IID): 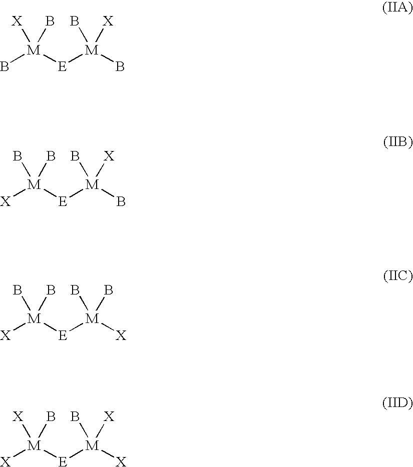
wherein:
each X is independently selected and is a monodentate ligand, or, in the case of formula (IID), the two X groups coordinated to a M atom may each be a monodentate ligand or may together form a dicarboxylate bidentate ligand;
each B is independently selected and is a ligand coordinated to the M atom by a nitrogen atom having a lone pair of electrons;
E is a ligand coordinated to each M atom by a nitrogen atom having a lone pair of electrons; and
each M is independently selected from the group consisting of Pt(II), Pd(II) and Au(II).

61. The composition as claimed in claim 59, wherein the metal complex is a metal complex of formula (IIIA), (IIIB), (IIIC) or (IIID): 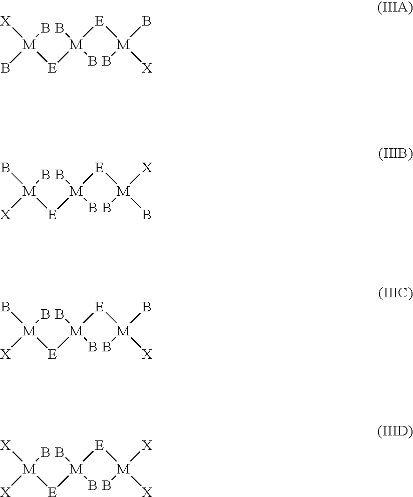
wherein:
each X is independently selected and is a monodentate ligand, or, in the case of formula (IIID), the two X groups coordinated to a M atom may each be a monodentate ligand or may together form a dicarboxylate bidentate ligand;
each B is independently selected and is a ligand coordinated to the M atom by a nitrogen atom having a lone pair of electrons;
each E is independently selected and is a ligand coordinated to each of two M atoms by a nitrogen atom having a lone pair of electrons; and
each M is independently selected from the group consisting of Pt(II), Pd(II) and Au(II).
